(19) United States
(12) Patent Application Publication
(10) Pub. No.: US 2017/0189500 A1
(43) Pub. Date: Jul. 6, 2017

(54) USE OF RECOMBINANT CLOSTRIDIAL NEUROTOXINS FOR THE TREATMENT OF PATIENTS HAVING CERTAIN MUSCLE-RELATED DISORDERS

(71) Applicant: MERZ PHARMA GMBH & CO. KGAA, Frankfurt am Main (DE)

(72) Inventors: Klaus FINK, Köln (DE); Susanne GRAFE, Düsseldorf (DE); Imke SCHULTZ, Rodgau (DE); Susanna ROLL, Stadecken-Elsheim (DE); Deryck JORDAN, Frankfurt am Main (DE)

(21) Appl. No.: 15/317,399

(22) PCT Filed: Jun. 12, 2015
(86) PCT No.: PCT/EP2015/001189

§ 371 Date: Dec. 8, 2016

(30) Foreign Application Priority Data
Jun. 13, 2014 (EP) 14002043.9

(51) Int. Cl. A61K 38/48; C12N 9/52

(52) U.S. Cl. CPC A61K 38/4893; C12Y 304/24069; C12N 9/52

(57) ABSTRACT

This invention relates to novel uses of recombinant clostridial neurotoxins exhibiting decreased duration of effect, in particular uses for the treatment of patients having experienced muscle-related disorders and/or injuries.

### FIELD OF THE INVENTION

This invention relates to novel uses of recombinant clostridial neurotoxins exhibiting decreased duration of effect, in particular uses for the treatment of patients suffering from muscle-related disorders and/or injuries.

### BACKGROUND OF THE INVENTION

Clostridium is a genus of anaerobe gram-positive bacteria, belonging to the Firmicutes. Clostridium consists of around 100 species that include common free-living bacteria as well as important pathogens, such as Clostridium botulinum and Clostridium tetani. Both species produce neurotoxins, botulinum toxin and tetanus toxin, respectively. These neurotoxins are potent inhibitors of calcium-dependent neurotransmitter secretion of neuronal cells and are among the strongest toxins known to man. The lethal dose in humans lies between 0.1 ng and 1 ng per kilogram of body weight.

Oral ingestion of botulinum toxin via contaminated food or generation of botulinum toxin in wounds can cause botulism, which is characterised by paralysis of various muscles. Paralysis of the breathing muscles can cause death of the affected individual.

Although both botulinum neurotoxin (BoNT) and tetanus neurotoxin (TeNT) function via a similar initial physiological mechanism of action, inhibiting neurotransmitter release from the axon of the affected neuron into the synapse, they differ in their clinical response. While the botulinum toxin acts at the neuromuscular junction and other cholinergic synapses in the peripheral nervous system, inhibiting the release of the neurotransmitter acetylcholine and thereby causing flaccid paralysis, the tetanus toxin, which is transcytoseed into sentral neurons, acts mainly in the central nervous system, preventing the release of the inhibitory neurotransmitters GABA (gamma-aminobutyric acid) and glycine by degrading the protein synaptobrevin. The consequent overactivity of spinal cord motor neurons causes generalized contractions of the agonist and antagonist musculature, termed a tetanic spasm (rigid paralysis).

While the tetanus neurotoxin exists in one immunologically distinct type, the botulinum neurotoxins are known to occur in seven different immunogenic serotypes, termed BoNT/A through BoNT/H with further subtypes. Most Clostridium botulinum strains produce one type of neurotoxin, but strains producing multiple toxins have also been described.

Botulinum and tetanus neurotoxins have highly homologous amino acid sequences and show a similar domain structure. Their biologically active form comprises two peptide chains, a light chain of about 50 kDa and a heavy chain of about 100 kDa, linked by a disulfide bond. A linker or loop region, whose length varies among different clostridial toxins, is located between the two cysteine residues forming the disulfide bond. This loop region is proteolytically cleaved by an unknown clostridial endoprotease to obtain the biologically active toxin.

The molecular mechanism of intoxication by TeNT and BoNT appears to be similar as well: entry into the target neuron is mediated by binding of the C-terminal part of the heavy chain to a specific cell surface receptor; the toxin is then taken up by receptor-mediated endocytosis. The low pH in the so formed endosome then triggers a conformational change in the clostridial toxin which allows it to embed itself in the endosomal membrane and to translocate through the endosomal membrane into the cytoplasm, where the disulfide bond joining the heavy and the light chain is reduced. The light chain can then selectively cleave so called SNARE-proteins, which are essential for different steps of neurotransmitter release into the synaptic cleft, e.g. recognition, docking and fusion of neurotransmitter-containing vesicles with the plasma membrane. TeNT, BoNT/B, BoNT/D, BoNT/F, and BoNT/G cause proteolytic cleavage of synaptobrevin or VAMP (vesicle-associated membrane protein), BoNT/A and BoNT/E cleave the plasma membrane-associated protein SNAP-25, and BoNT/C cleaves the integral plasma membrane protein syntaxin and SNAP-25.

In Clostridium botulinum, the botulinum toxin is formed as a protein complex comprising the neurotoxic component and non-toxic proteins. The accessory proteins embed the neurotoxic component thereby protecting it from degradation by digestive enzymes in the gastrointestinal tract. Thus, botulinum neurotoxins of most serotypes are orally toxic. Complexes with, for example, 450 kDa or with 900 kDa are obtainable from cultures of Clostridium botulinum.

In recent years, botulinum neurotoxins have been used as therapeutic agents, for example in the treatment of dystonias and spasms, and have additionally been used in cosmetic applications, such as the treatment of fine wrinkles. Preparations comprising botulinum toxin complexes are commercially available, e.g. from Ipsen Ltd (Dysport®) or Allergan Inc. (Botox®). A high purity neurotoxic component, free of any complexing proteins, is for example available from Merz Pharmaceuticals GmbH, Frankfurt (Xeomin®).

Clostridial neurotoxins are usually injected into the affected muscle tissue, bringing the agent close to the neuromuscular end plate, i.e. close to the cellular receptor mediating its uptake into the nerve cell controlling said affected muscle. Various degrees of neurotoxin spread have been observed. The neurotoxin spread is thought to depend on the injected amount and the particular neurotoxin preparation. It can result in adverse side effects such as paralysis in nearby muscle tissue, which can largely be avoided by reducing the injected doses to the therapeutically relevant level. Overdosing can also trigger the immune system to generate neutralizing antibodies that inactivate the neurotoxin preventing it from relieving the involuntary muscle activity. Immunologic tolerance to botulinum toxin has been shown to correlate with cumulative doses.

Clostridial neurotoxins display variable durations of action that are serotype specific. The clinical therapeutic effect of BoNT/A lasts approximately 3 months for neuromuscular disorders and 6 to 12 months for hyperhidrosis. The effects of BoNT/E, on the other hand, last about 4 weeks. One possible explanation for the divergent durations of action might be the distinct subcellular localizations of BoNT serotypes. The protease domain of BoNT/A light chain localizes in a punctate manner to the plasma membrane of neuronal cells, co-localizing with its substrate SNAP-25. In contrast, the short-duration BoNT/E serotype is cytoplasmic. Membrane association might protect BoNT/A from cytosolic degradation mechanisms allowing for prolonged persistence of BoNT/A in the neuronal cell.

The longer lasting therapeutic effect of BoNT/A makes it preferable for certain clinical uses and in particular for certain cosmetic uses compared to the other serotypes, for example serotypes B, C₁, D, E, F, G and H. On the other hand, it might be advantageous in certain scenarios to decrease the duration of the therapeutic effect of a botulinum neurotoxin in order to reduce the duration of muscle paralysis.

WO 2011/000929 and WO 2013/068476 describe neurotoxins exhibiting a shortened biological activity. In brief, the applications describe polypeptides comprising at least one E3 ligase recognition motif in the light chain, wherein said E3 ligase recognition motif is preferably a binding motif for the E3 ligase MDM2. Section [0006] of WO 2013/068476 generically lists a number of indications, which could potentially benefit from the application of modified neurotoxins with decreased duration of effect.

In particular, WO 2013/068476 describes variants of BoNT/E (SEQ ID NOs: 52 and 80 in WO 2013/068476), which were shown to have a duration of effect, which was decreased by about 25% compared to wild-type BoNT/E in a cell culture assay.

Despite the progress that has been made in the past in the treatment of indications that benefit from the intermittent paralysis of muscles, there is still a strong demand to further improve the therapeutic options available to the practitioner in the art, in particular in light of the fact that it might be desirable in certain indications, after an initial requirement for paralysing one or more muscles in such indication, to achieve an earlier recovery of muscle activity to assist the patient being treated in getting back to his or her normal life. To date, such aspects have not been addressed satisfactorily.

### OBJECTS OF THE INVENTION

It was an object of the invention to provide novel uses for recombinant clostridial neurotoxins exhibiting a decreased duration of effect, in particular uses for the treatment of patients suffering from muscle-related disorders and/or injuries, e.g. uses in support of physiotherapy and for improvement of healing after surgery, and in particular the use in the treatment of a patient having a flat foot deformity.

### SUMMARY OF THE INVENTION

The naturally occurring botulinum toxin serotypes display highly divergent durations of effect, with BoNT/A exhibiting the longest persistence, and BoNT/E exhibiting a comparatively short persistence. In order to broaden the applicability of botulinum neurotoxins, variants of BoNT/E have been created that exhibit a shorter duration of effect (see in particular WO 2013/068476).

Surprisingly, it has been identified that the variants disclosed in WO 2013/068476 might advantageously be used in particular situations, for which no satisfactory solution has been available so far.

Thus, the present invention relates to a botulinum neurotoxin subtype E with reduced persistence having a sequence according to SEQ ID NO: 1, or SEQ ID NO: 2, or a functionally active variant thereof, for use in the treatment of a patient, wherein the patient is suffering from a medical condition or overexertion, or has undergone a therapeutic intervention, wherein said medical condition, overexertion or said therapeutic intervention, respectively, results in a muscular disorder and/or injury that requires paralysis of the muscle or muscles involved in said disorder and/or injury, in particular wherein said medical condition or overexertion or said therapeutic intervention mandates a full physiotherapeutic treatment of said patient within less than four weeks.

In a second aspect, the present invention relates to method for the treatment of a patient, who is suffering from a medical condition or overexertion, or who has undergone a therapeutic intervention, wherein said medical condition, overexertion, or said therapeutic intervention, respectively, results in a muscular disorder and/or injury that requires paralysis of the muscle or muscles involved in said disorder and/or injury, particularly wherein said medical condition, overexertion or said therapeutic intervention mandates a full physiotherapeutic treatment of said patient within less than four weeks a tendon-related disorder and/or injury, comprising the step of administering a botulinum neurotoxin subtype E with reduced persistence having a sequence according to SEQ ID NO: 1, or SEQ ID NO: 2, or a functionally active variant thereof, to said patient.

### DETAILED DESCRIPTION OF THE INVENTION

The present invention may be understood more readily by reference to the following detailed description of the invention and the examples included therein.

Thus, the present invention relates to a botulinum neurotoxin subtype E with reduced persistence having a sequence according to SEQ ID NO: 1, or SEQ ID NO: 2 or a functionally active variant thereof, for use in the treatment of a patient, wherein the patient is suffering from a medical condition or overexertion, or has undergone a therapeutic intervention, wherein said medical conditions, or said therapeutic intervention, respectively, results in a muscular disorder and/or injury that requires paralysis of the muscle or muscles involved in said disorder and/or injury, in particular wherein said medical condition or overexertion, or said therapeutic intervention, mandates a full physiotherapeutic treatment of said patient within less than four weeks.

In a second aspect, the present invention relates to method for the treatment of a patient, who is suffering from a medical condition or overexertion, or who has undergone a therapeutic intervention, wherein said medical condition or overexertion, or said therapeutic intervention, respectively, results in a muscular disorder and/or injury that requires paralysis of the muscle or muscles involved in said disorder and/or injury, particularly wherein said medical condition or overexertion, or said therapeutic intervention, mandates a full physiotherapeutic treatment of said patient within less than four weeks a tendon-related disorder and/or injury, comprising the step of administering a botulinum neurotoxin subtype E with reduced persistence having a sequence according to SEQ ID NO: 1, or SEQ ID NO: 2, or a functionally active variant thereof, to said patient.

In the context of the present invention, the term “functionally active variant” refers to a neurotoxin, in particular a recombinant neurotoxin, that differs in the amino acid sequence and/or the nucleic acid sequence encoding the amino acid sequence from the botulinum neurotoxin subtype E with reduced persistence having a sequence according to SEQ ID NO: 1 or SEQ ID NO: 2, but is still functionally active. In the context of the present invention, the term “functionally active” refers to the property of such recombinant clostridial neurotoxin variant to (i) achieve muscle paralysis to at least 50%, particularly to at least 60%, at least 70%, at least 80%, and most particularly at least 90% of the muscle paralysis achieved with the same amount of a botulinum neurotoxin subtype E with reduced persistence having a sequence according to SEQ ID NO: 1 or SEQ ID NO: 2, and (ii) achieve such muscle paralysis for a duration of time that is at maximum 10% shorter or longer, particularly at maximum 5% shorter or longer than the duration of paralysis achieved by a botulinum neurotoxin subtype E with reduced persistence having a sequence according to SEQ ID NO: 1 or SEQ ID NO: 2 (i.e. which shows between 90% and 110% of the duration of paralysis, particularly between 95% and 105% of the duration of paralysis achieved by a botulinum neurotoxin subtype E with reduced persistence having a sequence according to SEQ ID NO: 1 or SEQ ID NO: 2).

On the protein level, a functionally active variant will maintain key features of the corresponding parental clostridial neurotoxin, such as key residues for the endopeptidase activity in the light chain, or key residues for the attachment to the neurotoxin receptors or for translocation through the endosomal membrane in the heavy chain, but may contain one or more mutations comprising a deletion of one or more amino acids of the corresponding clostridial neurotoxin, an addition of one or more amino acids of the corresponding clostridial neurotoxin, and/or a substitution of one or more amino acids of the corresponding clostridial neurotoxin. Particularly, said deleted, added and/or substituted amino acids are consecutive amino acids. According to the teaching of the present invention, any number of amino acids may be added, deleted, and/or substituted, as long as the functionally active variant remains biologically active as defined above. For example, 1, 2, 3, 4, 5, up to 10, up to 15, up to 25, up to 50, up to 100, up to 200, up to 400, up to 500 amino acids or even more amino acids of a botulinum neurotoxin subtype E with reduced persistence having a sequence according to SEQ ID NO: 1 or SEQ ID NO: 2 may be added, deleted, and/or substituted. This neurotoxin fragment may contain an N-terminal, C-terminal, and/or one or more internal deletion(s).

In another embodiment, the functional variant of a clostridial neurotoxin additionally comprises a signal peptide. Usually, said signal peptide will be located at the N-terminus of the neurotoxin. Many such signal peptides are known in the art and are comprised by the present invention. In particular, the signal peptide results in transport of the neurotoxin across a biological membrane, such as the membrane of the endoplasmic reticulum, the Golgi membrane or the plasma membrane of a eukaryotic or prokaryotic cell. It has been found that signal peptides, when attached to the neurotoxin, will mediate secretion of the neurotoxin into the supernatant of the cells. In certain embodiments, the signal peptide will be cleaved off in the course of, or subsequent to, secretion, so that the secreted protein lacks the N-terminal signal peptide, is composed of separate light and heavy chains, which are covalently linked by disulfide bridges, and is proteolytically active.

In particular embodiments, the functional variant has in its clostridium neurotoxin part a sequence identity of at least 40%, at least 50%, at least 60%, at least 70% or most particularly at least 80%, and a sequence homology of at least 60%, at least 70%, at least 80%, at least 90%, or most particularly at least 95% to the corresponding part of a botulinum neurotoxin subtype E with reduced persistence having a sequence according to SEQ ID NO: 1 or SEQ ID NO: 2. Methods and algorithms for determining sequence identity and/or homology, including the comparison of variants having deletions, additions, and/or substitutions relative to a parental sequence, are well known to the practitioner of ordinary skill in the art. On the DNA level, the nucleic acid sequences encoding the functional homologue and the parental clostridial neurotoxin may differ to a larger extent due to the degeneracy of the genetic code. It is known that the usage of codons is different between prokaryotic and eukaryotic organisms. Thus, when expressing a prokaryotic protein such as a clostridial neurotoxin, in a eukaryotic expression system, it may be necessary, or at least helpful, to adapt the nucleic acid sequence to the codon usage of the expression host cell, meaning that sequence identity or homology may be rather low on the nucleic acid level.

In the context of the present invention, the term “variant” refers to a neurotoxin that is a chemically, enzymatically, or genetically modified derivative of a botulinum neurotoxin subtype E with reduced persistence having a sequence according to SEQ ID NO: 1 or SEQ ID NO: 2. A chemically modified derivative may be one that is modified by pyruvation, phosphorylation, sulfatation, lipidation, pegylation, glycosylation and/or the chemical addition of an amino acid or a polypeptide comprising between 2 and 100 amino acids, including modification occurring in the eukaryotic host cell used for expressing the derivative. An enzymatically modified derivative is one that is modified by the activity of enzymes, such as endo- or exoproteolytic enzymes, including modification by enzymes of the eukaryotic host cell used for expressing the derivative. As pointed out above, a genetically modified derivative is one that has been modified by deletion or substitution of one or more amino acids contained in, or by addition of one or more amino acids (including polypeptides comprising between 2 and about 100 amino acids) to, the amino acid sequence of said clostridial neurotoxin. Methods for designing and constructing such chemically or genetically modified derivatives and for testing of such variants for functionality are well known to anyone of ordinary skill in the art.

In the context of the present invention, the term “recombinant neurotoxin” refers to a composition comprising a clostridial neurotoxin that is obtained by expression of the neurotoxin in a heterologous cell such as E. coli, and including, but not limited to, the raw material obtained from a fermentation process (supernatant, composition after cell lysis), a fraction comprising a clostridial neurotoxin obtained from separating the ingredients of such a raw material in a purification process, an isolated and essentially pure protein, and a formulation for pharmaceutical and/or aesthetic use comprising a clostridial neurotoxin and additionally pharmaceutically acceptable solvents and/or excipients.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components, or groups thereof. The term “comprising” thus includes the more restrictive terms “consisting of” and “consisting essentially of”.

In the context of the present invention, the term “botulinum neurotoxin subtype E” refers to a particular neurotoxin found in and obtainable from Clostridium botulinum having a sequence shown in SEQ ID NO: 82 of WO 2013/068476.

In particular embodiments, said functionally active variant has a persistence that is at maximum 5% shorter or longer than the duration of paralysis achieved by a botulinum neurotoxin subtype E with reduced persistence having a sequence according to SEQ ID NO: 1 or SEQ ID NO: 2.

Without wishing to be bound by theory, the recombinant clostridial neurotoxins of the present invention might show decreased biological half-life, increased degradation rates, increased diffusion rates, decreased uptake by neuronal cells, and/or modified intracellular translocation rates, in each case relative to wild-type botulinum neurotoxin of subtype E (BoNT/E).

In certain medical conditions, or in the case of a patient having undergone a therapeutic intervention, muscular disorders such as overexertion (muscle fatigue), muscle tightness, increased and/or imbalanced muscle tonus muscle contraction, muscle cramps, acute spasms, muscle contracture, a muscle imbalance between an agonist and an antagonist muscle, and pathologies resulting therefrom, may result that are very painful, and that may lead to inflammatory processes in tendons and fascies and malposition of joints (Barrett: Podiatry Today Volume 24—Issue 5—May 2011; DiGiovanni, et al, j Bone Joint surg Am, 2002 June; 84(6):962-970; Bowers & Castro, The mechanics behind the image: foot and ankle pathology associated with gastrocnemius contracture, Semin Musculoskelet Radiol. 2007 March; 11(1)L83-90).

Particularly, muscles may be affected that are selected from the list of: hamstrings, gastrocnemius, triceps and quadriceps.

In particular embodiments, muscles of the elbow may be affected. Elbow flexion contractures can have traumatic causes following injury or surgery or atraumatic causes as osteoarthritis. The hypertonic elbow flexors impede elbow extension causing a stiff elbow. Particularly, muscles may be affected that are selected from the list of: Elbow flexors, M bracialis, M. biceps brachii, M. brachioradialis, and M. pronator teres.

Similarly, muscle injuries, such as muscle strains or tears, or wound affecting a muscle may result in pain, and movements involving the affected muscle may delay wound healing.

Permanent immobilization of the muscles involved may relieve the patient afflicted with a muscular disorder and/or injury from the pain sensation, and may support, for example, the wound healing process. However, in many instances a full physiotherapeutic treatment of said patient may be necessary within less than four weeks from the start of treating a patient suffering from such medical condition or having experienced such therapeutic intervention. If physiotherapy is needed to regain full range of motion, a high compliance of the patient or an active assist approach will be chosen. This is very painful and without guarantee to get rid of the contracture. If an incidence of pathological muscle contracture occurs the first line standard-of-care is oral medication to relieve the pain, or using a systemic muscle relaxant. These may have considerable side effects and none of them are curative.

In certain embodiments, the patient is suffering from a tendinopathy.

In certain embodiments, the patient is suffering from muscle tightness or a pathological condition resulting therefrom. In particular embodiment, the patient is suffering from an imbalance in the muscles, with some muscles being overactive, and some being underactive, wherein treatment of the overactive, stronger muscle permits the underactive, weaker muscle to regains its normal function. Due to decreased duration of effect, atrophy of the treated muscle is prohibited.

In certain embodiments, the patient is suffering from a foot or ankle pathology particularly a pathology caused by a contracture of the musculus gastrocnemius and/or the musculus soleus, which results in a limited range of movement of the ankle and for example in a pes equinus, which in turn causes overexertion of the tendons and muscles of the central and front foot and a number of pathologies resulting therefrom. In certain embodiments, the patient is suffering from a debilitating foot condition, in particular selected from the list of flat foot, metatarsalgia, capsulitis, plantar fasciitis, hallux valgus, curled fifth toe, pes equinus, sesamoiditis, diabetic foot ulcers, Achilles tendon inflammation, tendinitis, Achilles tendon rupture, pes equinus, pes cavus, hammer toe, idiopathic toe walking and charcot athropathy. In certain embodiments, the musculus gastrocnemius, musculus triceps surae and/or musculus soleus is short or tight which leads to one of the above mentioned foot problems. Relaxing the muscle(s) with a short acting neurotoxin relieves pain and enables stretching and physiotherapy without weakening the muscle for a too long time, thus preventing muscle atrophy.

In certain embodiments, the patient is suffering from a muscle strain or muscle tear. In certain such embodiments, the patient is suffering muscle spasms, which reduce blood flow and cause accumulation of metabolic products, which in turn may result in pain. In certain embodiments, the muscle is a muscle of the upper or lower leg.

Acute to sub-acute medical needs resulting from inappropriate or pathological involuntary muscle contraction or by muscle contracture, especially in the physiatrist/physical medicine and rehabilitation field can ideally be addressed by a short acting and faster-onset molecule for paralysis of the muscle or muscles involved in said disorder. The proteins having a sequence according to SEQ ID NO: 1 or 2 will enable an earlier start of passive and active motion (already after 4+/−2 weeks) after surgery or incident, therewith shorten the stay in rehabilitation, shorten the stay at home and away from work. Such treatment will avoid atrophy caused by a long paralysis and presumably lower pain associated with these conditions.

For the medical needs mentioned above in acute to sub-acute care it is mandatory to transfer very fast from an immobilised phase to activity. After surgery or incidence three phases of regaining full range of motion, necessary for long-term improvement of the treatment can be described.

Phase 1 (Approx. Week 1 to 4):
Immobilization and/or passive range of motion exercise phase, for which the full neurotoxin effect is needed.

Phase 2 (Week 5 to 8):
Mild exercise: active assist and active range of motion exercises are performed for which the effect of the neurotoxin should be gone or could be waning.

Phase 3 (Weeks 9 to 12):
Exercise phase in which the active range of motion exercise has to be performed for which the neurotoxin effect must be gone.

From the time lines it becomes obvious that the duration of effect of BoNT/A interferes with the physical therapy during orthopaedic rehabilitation whereas the duration of effect of the proteins having a sequence according to SEQ ID NO: 1 or 2 will enable to perform the needed exercises.

Thus, since the proteins having a sequence according to SEQ ID NO: 1 or 2 have a shorter duration of effect, a relaxation of the affected muscles lasts only 2 to 4 weeks. Patient can perform physiotherapy earlier without the risk of long-paralysis-induced muscle atrophy. Increase of muscle size and strength for stabilization of the affected joint can be accomplished earlier in comparison to a botulinum toxin of serotype A with a duration of 12 weeks or even in comparison to wild-type botulinum toxin of serotype E. As a result of earlier muscle regeneration a reduction of overall physiotherapy is expected since the patient has its ability to walk earlier. Furthermore, the duration of botulinum toxin-specific side effects will be reduced due to the shorter duration of the toxin. Thus, one or more of the following advantages can be accomplished:
muscle atrophy can be reduced
pain caused by pathological muscle contracture can be reduced
pain medication can be reduced
enablement of early physiotherapy increases chance to regain full range of motion
consecutive incidences or surgery can be avoided
development of a permanent disabling status can be avoided
amount of physiotherapy can be decreased
surgery with relaxed muscles can be enabled
quality of life can be increased
ability to work can be increased

In particular embodiments, said medical condition is selected from the list of: (a) pre- or post-surgical local muscle relaxation that improves or accelerates restitution, (b) muscle contracture or contractions unrelated to surgery that benefit from transient local relaxation, (c) pathologic muscular of connective tissue conditions that can be treated only or earlier by active or passive physical therapy; and (d) “overexertion”.

Additionally, particularly in the situation of a patient having undergone surgery, it may be mandatory to prevent unnecessary or excessive movement of muscles in close proximity to the site of surgical intervention. This is particularly the case in patients with Parkinson's disease or any other disease or disorder accompanied by involuntary muscle movements, such as tremor.

Thus, in particular embodiments, the patient is a patient suffering from involuntary muscle movements associated with a disease, in particular Parkinson's disease or any other disease or disorder accompanied by involuntary muscle movements, such as tremor

In particular embodiments, muscle paralysis by a botulinum neurotoxin of more than 5 weeks, in particular of more than 4 weeks, and more particularly of more than 3 weeks, is contraindicated and/or deemed to be associated with negative impact on overall treatment success, particularly due to high likelihood of increased muscle atrophy.

### EXAMPLES

### Example 1: Treatment of a Patient with Flat Foot Deformity

A patient has flat foot deformity of the triceps surae muscle with contracture and a corresponding reduction in dorsiflexion of the ankle. An injection of the protein having a sequence according to SEQ ID NO: 1 in the triceps surae muscle allows performing stretching physiotherapy without pain within one day. After two weeks the patient can perform all activities and the ankle stiffness is significantly reduced.

### Example 2: Treatment of a Patient with Plantar Fasciitis

Plantar fasciitis is a common cause of heel pain in adults. In plantar fasciitis a strong correlation between the degree of gastrocnemius tightness and pain has been found. An injection of recombinant neurotoxin in the triceps surae will relief pain by reducing muscle tightness and allow healing process. Stretching and strengthening exercises can further reduce tension and stress on the foot and improve foot mechanics.

SEQUENCES

```
SEQ ID NO. 1
Met Pro Lys Ile Asn Ser Phe Asn Tyr Asn Asp Pro Val Asn Asp Arg
1 5 10 15
Thr Ile Leu Tyr Ile Lys Pro Gly Gly Cys Gln Glu Phe Tyr Lys Ser
                        20 25 30
Phe Asn Ile Met Lys Asn Ile Trp Ile Ile Pro Glu Arg Asn Val Ile
                35 40 45
Gly Thr Thr Pro Gln Asp Phe His Pro Pro Thr Ser Leu Lys Asn Gly
        50 55 60
Asp Ser Ser Tyr Tyr Asp Pro Asn Tyr Leu Gln Ser Asp Glu Glu Lys
65 70 75 80
Asp Arg Phe Leu Lys Ile Val Thr Lys Ile Phe Asn Arg Ile Asn Asn
                                85 90 95
Asn Leu Ser Gly Gly Ile Leu Leu Glu Glu Leu Ser Lys Ala Asn Pro
                        100 105 110
Tyr Leu Gly Asn Asp Asn Thr Pro Asp Asn Gln Phe His Ile Gly Asp
                115 120 125
Ala Ser Ala Val Glu Ile Lys Phe Ser Asn Gly Ser Gln Asp Ile Leu
        130 135 140
Leu Pro Asn Val Ile Ile Met Gly Ala Glu Pro Asp Leu Phe Glu Thr
145 150 155 160
Asn Ser Ser Asn Ile Ser Leu Arg Asn Asn Tyr Met Pro Ser Asn His
                                165 170 175
Gly Phe Gly Ser Ile Ala Ile Val Thr Phe Ser Pro Glu Tyr Ser Phe
                        180 185 190
Arg Phe Asn Asp Asn Ser Met Asn Glu Phe Ile Gln Asp Pro Ala Leu
                195 200 205
Thr Leu Met His Glu Leu Ile His Ser Leu His Gly Leu Tyr Gly Ala
        210 215 220
Lys Gly Ile Thr Thr Lys Tyr Thr Ile Thr Gln Lys Gln Asn Pro Leu
225 230 235 240
Ile Thr Asn Ile Arg Gly Thr Asn Ile Glu Glu Phe Leu Thr Phe Gly
                                245 250 255
Gly Thr Asp Leu Asn Ile Ile Thr Ser Ala Gln Ser Asn Asp Ile Tyr
                        260 265 270
Thr Asn Leu Leu Ala Asp Tyr Lys Lys Ile Ala Ser Lys Leu Ser Lys
                275 280 285
Val Gln Val Ser Asn Pro Leu Leu Asn Pro Tyr Lys Asp Val Phe Glu
        290 295 300
Ala Lys Tyr Gly Leu Asp Lys Asp Ala Ser Gly Ile Tyr Ser Val Asn
305 310 315 320
Ile Asn Lys Phe Asn Asp Ile Phe Lys Lys Leu Tyr Ser Phe Thr Glu
                                325 330 335
Phe Asp Leu Ala Thr Lys Phe Gln Val Lys Cys Arg Gln Thr Tyr Ile
                        340 345 350
Gly Gln Tyr Lys Tyr Phe Lys Leu Ser Asn Leu Leu Asn Asp Ser Ile
                355 360 365
Tyr Asn Ile Ser Glu Gly Tyr Asn Ile Asn Asn Leu Lys Val Asn Phe
        370 375 380
Arg Gly Gln Asn Ala Asn Leu Asn Pro Arg Ile Ile Thr Pro Ile Thr
385 390 395 400
Gly Arg Gly Leu Val Lys Lys Ile Ile Arg Phe Cys Val Arg Gly Ile
                                405 410 415
Ile Thr Ser Leu Thr Phe Glu His Asn Trp Ala Gln Leu Glu Asn Lys
                        420 425 430
Ser Leu Val Pro Arg Gly Ser Lys Ala Leu Asn Asp Leu Cys Ile Glu
                435 440 445
Ile Asn Asn Gly Glu Leu Phe Phe Val Ala Ser Glu Asn Ser Tyr Asn
        450 455 460
Asp Asp Asn Ile Asn Thr Pro Lys Glu Ile Asp Asp Thr Val Thr Ser
465 470 475 480
Asn Asn Asn Tyr Glu Asn Asp Leu Asp Gln Val Ile Leu Asn Phe Asn
                                485 490 495
Ser Glu Ser Ala Pro Gly Leu Ser Asp Glu Lys Leu Asn Leu Thr Ile
                        500 505 510
Gln Asn Asp Ala Tyr Ile Pro Lys Tyr Asp Ser Asn Gly Thr Ser Asp
                515 520 525
Ile Glu Gln His Asp Val Asn Glu Leu Asn Val Phe Phe Tyr Leu Asp
        530 535 540
Ala Gln Lys Val Pro Glu Gly Glu Asn Asn Val Asn Leu Thr Ser Ser
545 550 555 560
Ile Asp Thr Ala Leu Leu Glu Gln Pro Lys Ile Tyr Thr Phe Phe Ser
                                565 570 575
Ser Glu Phe Ile Asn Asn Val Asn Lys Pro Val Gln Ala Ala Leu Phe
                        580 585 590
Val Ser Trp Ile Gln Gln Val Leu Val Asp Phe Thr Thr Glu Ala Asn
                595 600 605
Gln Lys Ser Thr Val Asp Lys Ile Ala Asp Ile Ser Ile Val Val Pro
        610 615 620
Tyr Ile Gly Leu Ala Leu Asn Ile Gly Asn Glu Ala Gln Lys Gly Asn
625 630 635 640
Phe Lys Asp Ala Leu Glu Leu Leu Gly Ala Gly Ile Leu Leu Glu Phe
                                645 650 655
Glu Pro Glu Leu Leu Ile Pro Thr Ile Leu Val Phe Thr Ile Lys Ser
                        660 665 670
Phe Leu Gly Ser Ser Asp Asn Lys Asn Lys Val Ile Lys Ala Ile Asn
                675 680 685
Asn Ala Leu Lys Glu Arg Asp Glu Lys Trp Lys Glu Val Tyr Ser Phe
        690 695 700
Ile Val Ser Asn Trp Met Thr Lys Ile Asn Thr Gln Phe Asn Lys Arg
705 710 715 720
Lys Glu Gln Met Tyr Gln Ala Leu Gln Asn Gln Val Asn Ala Ile Lys
                                725 730 735
Thr Ile Ile Glu Ser Lys Tyr Asn Ser Tyr Thr Leu Glu Glu Lys Asn
                        740 745 750
Glu Leu Thr Asn Lys Tyr Asp Ile Lys Gln Ile Glu Asn Glu Leu Asn
                755 760 765
Gln Lys Val Ser Ile Ala Met Asn Asn Ile Asp Arg Phe Leu Thr Glu
        770 775 780
Ser Ser Ile Ser Tyr Leu Met Lys Leu Ile Asn Glu Val Lys Ile Asn
785 790 795 800
Lys Leu Arg Glu Tyr Asp Glu Asn Val Lys Thr Tyr Leu Leu Asn Tyr
                                805 810 815
Ile Ile Gln His Gly Ser Ile Leu Gly Glu Ser Gln Gln Glu Leu Asn
                        820 825 830
Ser Met Val Thr Asp Thr Leu Asn Asn Ser Ile Pro Phe Lys Leu Ser
                835 840 845
Ser Tyr Thr Asp Asp Lys Ile Leu Ile Ser Tyr Phe Asn Lys Phe Phe
        850 855 860
Lys Arg Ile Lys Ser Ser Ser Val Leu Asn Met Arg Tyr Lys Asn Asp
865 870 875 880
Lys Tyr Val Asp Thr Ser Gly Tyr Asp Ser Asn Ile Asn Ile Asn Gly
                                885 890 895
Asp Val Tyr Lys Tyr Pro Thr Asn Lys Asn Gln Phe Gly Ile Tyr Asn
                        900 905 910
Asp Lys Leu Ser Glu Val Asn Ile Ser Gln Asn Asp Tyr Ile Ile Tyr
                915 920 925
Asp Asn Lys Tyr Lys Asn Phe Ser Ile Ser Phe Trp Val Arg Ile Pro
        930 935 940
Asn Tyr Asp Asn Lys Ile Val Asn Val Asn Asn Glu Tyr Thr Ile Ile
945 950 955 960
Asn Cys Met Arg Asp Asn Asn Ser Gly Trp Lys Val Ser Leu Asn His
                                965 970 975
Asn Glu Ile Ile Trp Thr Leu Gln Asp Asn Ala Gly Ile Asn Gln Lys
                        980 985 990
Leu Ala Phe Asn Tyr Gly Asn Ala Asn Gly Ile Ser Asp Tyr Ile Asn
                995 1000 1005
Lys Trp Ile Phe Val Thr Ile Thr Asn Asp Arg Leu Gly Asp Ser Lys
        1010 1015 1020
Leu Tyr Ile Asn Gly Asn Leu Ile Asp Gln Lys Ser Ile Leu Asn Leu
1025 1030 1035 1040
Gly Asn Ile His Val Ser Asp Asn Ile Leu Phe Lys Ile Val Asn Cys
                                1045 1050 1055
Ser Tyr Thr Arg Tyr Ile Gly Ile Arg Tyr Phe Asn Ile Phe Asp Lys
                        1060 1065 1070
Glu Leu Asp Glu Thr Glu Ile Gln Thr Leu Tyr Ser Asn Glu Pro Asn
                1075 1080 1085
Thr Asn Ile Leu Lys Asp Phe Trp Gly Asn Tyr Leu Leu Tyr Asp Lys
        1090 1095 1100
Glu Tyr Tyr Leu Leu Asn Val Leu Lys Pro Asn Asn Phe Ile Asp Arg
1105 1110 1115 1120
Arg Lys Asp Ser Thr Leu Ser Ile Asn Asn Ile Arg Ser Thr Ile Leu
                                1125 1130 1135
Leu Ala Asn Arg Leu Tyr Ser Gly Ile Lys Val Lys Ile Gln Arg Val
                        1140 1145 1150
Asn Asn Ser Ser Thr Asn Asp Asn Leu Val Arg Lys Asn Asp Gln Val
                1155 1160 1165
Tyr Ile Asn Phe Val Ala Ser Lys Thr His Leu Phe Pro Leu Tyr Ala
        1170 1175 1180
Asp Thr Ala Thr Thr Asn Lys Glu Lys Thr Ile Lys Ile Ser Ser Ser
1185 1190 1195 1200
Gly Asn Arg Phe Asn Gln Val Val Val Met Asn Ser Val Gly Asn Asn
                                1205 1210 1215
Cys Thr Met Asn Phe Lys Asn Asn Asn Gly Asn Asn Ile Gly Leu Leu
                        1220 1225 1230
Gly Phe Lys Ala Asp Thr Val Val Ala Ser Thr Trp Tyr Tyr Thr His
                1235 1240 1245
Met Arg Asp His Thr Asn Ser Asn Gly Cys Phe Trp Asn Phe Ile Ser
        1250 1255 1260
Glu Glu His Gly Trp Gln Glu Lys
1265 1270
SEQ ID NO. 2
Met Pro Lys Ile Asn Ser Phe Asn Tyr Asn Asp Pro Val Asn Asp Arg
1 5 10 15
Thr Ile Leu Tyr Ile Lys Pro Gly Gly Cys Gln Glu Phe Tyr Lys Ser
                        20 25 30
Phe Asn Ile Met Lys Asn Ile Trp Ile Ile Pro Glu Arg Asn Val Ile
                35 40 45
Gly Thr Thr Pro Gln Asp Phe His Pro Pro Thr Ser Leu Lys Asn Gly
        50 55 60
Asp Ser Ser Tyr Tyr Asp Pro Asn Tyr Leu Gln Ser Asp Glu Glu Lys
65 70 75 80
Asp Arg Phe Leu Lys Ile Val Thr Lys Ile Phe Asn Arg Ile Asn Asn
                                85 90 95
Asn Leu Ser Gly Gly Ile Leu Leu Glu Glu Leu Ser Lys Ala Asn Pro
                        100 105 110
Tyr Leu Gly Asn Asp Asn Thr Pro Asp Asn Gln Phe His Ile Gly Asp
                115 120 125
Ala Ser Ala Val Glu Ile Lys Phe Ser Asn Gly Ser Gln Asp Ile Leu
        130 135 140
Leu Pro Asn Val Ile Ile Met Gly Ala Glu Pro Asp Leu Phe Glu Thr
145 150 155 160
Asn Ser Ser Asn Ile Ser Leu Arg Asn Asn Tyr Met Pro Ser Asn His
                                165 170 175
Gly Phe Gly Ser Ile Ala Ile Val Thr Phe Ser Pro Glu Tyr Ser Phe
                        180 185 190
Arg Phe Asn Asp Asn Ser Met Asn Glu Phe Ile Gln Asp Pro Ala Leu
                195 200 205
Thr Leu Met His Glu Leu Ile His Ser Leu His Gly Leu Tyr Gly Ala
        210 215 220
Lys Gly Ile Thr Thr Lys Tyr Thr Ile Thr Gln Lys Gln Asn Pro Leu
225 230 235 240
Ile Thr Asn Ile Arg Gly Thr Asn Ile Glu Glu Phe Leu Thr Phe Gly
                                245 250 255
Gly Thr Asp Leu Asn Ile Ile Thr Ser Ala Gln Ser Asn Asp Ile Tyr
                        260 265 270
Thr Asn Leu Leu Ala Asp Tyr Lys Lys Ile Ala Ser Lys Leu Ser Lys
                275 280 285
Val Gln Val Ser Asn Pro Leu Leu Asn Pro Tyr Lys Asp Val Phe Glu
        290 295 300
Ala Lys Tyr Gly Leu Asp Lys Asp Ala Ser Gly Ile Tyr Ser Val Asn
305 310 315 320
Ile Asn Lys Phe Asn Asp Ile Phe Lys Lys Leu Tyr Ser Phe Thr Glu
                                325 330 335
Phe Asp Leu Ala Thr Lys Phe Gln Val Lys Cys Arg Gln Thr Tyr Ile
                        340 345 350
Gly Gln Tyr Lys Tyr Phe Lys Leu Ser Asn Leu Leu Asn Asp Ser Ile
                355 360 365
Tyr Asn Ile Ser Glu Gly Tyr Asn Ile Asn Asn Leu Lys Val Asn Phe
        370 375 380
Arg Gly Gln Asn Ala Asn Leu Asn Pro Arg Ile Ile Thr Pro Ile Thr
385 390 395 400
Gly Arg Gly Leu Val Lys Lys Ile Ile Arg Phe Cys Val Arg Gly Ile
                                405 410 415
Ile Thr Ser Leu Thr Phe Glu His Asn Trp Ala Gln Leu Thr Ser Lys
                        420 425 430
Ser Leu Val Pro Arg Gly Ser Lys Ala Leu Asn Asp Leu Cys Ile Glu
                435 440 445
Ile Asn Asn Gly Glu Leu Phe Phe Val Ala Ser Glu Asn Ser Tyr Asn
        450 455 460
Asp Asp Asn Ile Asn Thr Pro Lys Glu Ile Asp Asp Thr Val Thr Ser
465 470 475 480
Asn Asn Asn Tyr Glu Asn Asp Leu Asp Gln Val Ile Leu Asn Phe Asn
                                485 490 495
Ser Glu Ser Ala Pro Gly Leu Ser Asp Glu Lys Leu Asn Leu Thr Ile
                        500 505 510
Gln Asn Asp Ala Tyr Ile Pro Lys Tyr Asp Ser Asn Gly Thr Ser Asp
                515 520 525
Ile Glu Gln His Asp Val Asn Glu Leu Asn Val Phe Phe Tyr Leu Asp
        530 535 540
Ala Gln Lys Val Pro Glu Gly Glu Asn Asn Val Asn Leu Thr Ser Ser
545 550 555 560
Ile Asp Thr Ala Leu Leu Glu Gln Pro Lys Ile Tyr Thr Phe Phe Ser
                                565 570 575
Ser Glu Phe Ile Asn Asn Val Asn Lys Pro Val Gln Ala Ala Leu Phe
                        580 585 590
Val Ser Trp Ile Gln Gln Val Leu Val Asp Phe Thr Thr Glu Ala Asn
                595 600 605
Gln Lys Ser Thr Val Asp Lys Ile Ala Asp Ile Ser Ile Val Val Pro
        610 615 620
Tyr Ile Gly Leu Ala Leu Asn Ile Gly Asn Glu Ala Gln Lys Gly Asn
625 630 635 640
Phe Lys Asp Ala Leu Glu Leu Leu Gly Ala Gly Ile Leu Leu Glu Phe
                                645 650 655
Glu Pro Glu Leu Leu Ile Pro Thr Ile Leu Val Phe Thr Ile Lys Ser
                        660 665 670
Phe Leu Gly Ser Ser Asp Asn Lys Asn Lys Val Ile Lys Ala Ile Asn
                675 680 685
Asn Ala Leu Lys Glu Arg Asp Glu Lys Trp Lys Glu Val Tyr Ser Phe
        690 695 700
Ile Val Ser Asn Trp Met Thr Lys Ile Asn Thr Gln Phe Asn Lys Arg
705 710 715 720
Lys Glu Gln Met Tyr Gln Ala Leu Gln Asn Gln Val Asn Ala Ile Lys
                                725 730 735
Thr Ile Ile Glu Ser Lys Tyr Asn Ser Tyr Thr Leu Glu Glu Lys Asn
                        740 745 750
Glu Leu Thr Asn Lys Tyr Asp Ile Lys Gln Ile Glu Asn Glu Leu Asn
                755 760 765
Gln Lys Val Ser Ile Ala Met Asn Asn Ile Asp Arg Phe Leu Thr Glu
        770 775 780
Ser Ser Ile Ser Tyr Leu Met Lys Leu Ile Asn Glu Val Lys Ile Asn
785 790 795 800
Lys Leu Arg Glu Tyr Asp Glu Asn Val Lys Thr Tyr Leu Leu Asn Tyr
                                805 810 815
Ile Ile Gln His Gly Ser Ile Leu Gly Glu Ser Gln Gln Glu Leu Asn
                        820 825 830
Ser Met Val Thr Asp Thr Leu Asn Asn Ser Ile Pro Phe Lys Leu Ser
                835 840 845
Ser Tyr Thr Asp Asp Lys Ile Leu Ile Ser Tyr Phe Asn Lys Phe Phe
        850 855 860
Lys Arg Ile Lys Ser Ser Ser Val Leu Asn Met Arg Tyr Lys Asn Asp
865 870 875 880
Lys Tyr Val Asp Thr Ser Gly Tyr Asp Ser Asn Ile Asn Ile Asn Gly
                                885 890 895
Asp Val Tyr Lys Tyr Pro Thr Asn Lys Asn Gln Phe Gly Ile Tyr Asn
                        900 905 910
Asp Lys Leu Ser Glu Val Asn Ile Ser Gln Asn Asp Tyr Ile Ile Tyr
                915 920 925
Asp Asn Lys Tyr Lys Asn Phe Ser Ile Ser Phe Trp Val Arg Ile Pro
        930 935 940
Asn Tyr Asp Asn Lys Ile Val Asn Val Asn Asn Glu Tyr Thr Ile Ile
945 950 955 960
Asn Cys Met Arg Asp Asn Asn Ser Gly Trp Lys Val Ser Leu Asn His
                                965 970 975
Asn Glu Ile Ile Trp Thr Leu Gln Asp Asn Ala Gly Ile Asn Gln Lys
                        980 985 990
Leu Ala Phe Asn Tyr Gly Asn Ala Asn Gly Ile Ser Asp Tyr Ile Asn
                995 1000 1005
Lys Trp Ile Phe Val Thr Ile Thr Asn Asp Arg Leu Gly Asp Ser Lys
        1010 1015 1020
Leu Tyr Ile Asn Gly Asn Leu Ile Asp Gln Lys Ser Ile Leu Asn Leu
1025 1030 1035 1040
Gly Asn Ile His Val Ser Asp Asn Ile Leu Phe Lys Ile Val Asn Cys
                                1045 1050 1055
Ser Tyr Thr Arg Tyr Ile Gly Ile Arg Tyr Phe Asn Ile Phe Asp Lys
                        1060 1065 1070
Glu Leu Asp Glu Thr Glu Ile Gln Thr Leu Tyr Ser Asn Glu Pro Asn
                1075 1080 1085
Thr Asn Ile Leu Lys Asp Phe Trp Gly Asn Tyr Leu Leu Tyr Asp Lys
        1090 1095 1100
Glu Tyr Tyr Leu Leu Asn Val Leu Lys Pro Asn Asn Phe Ile Asp Arg
1105 1110 1115 1120
Arg Lys Asp Ser Thr Leu Ser Ile Asn Asn Ile Arg Ser Thr Ile Leu
                                1125 1130 1135
Leu Ala Asn Arg Leu Tyr Ser Gly Ile Lys Val Lys Ile Gln Arg Val
                        1140 1145 1150
Asn Asn Ser Ser Thr Asn Asp Asn Leu Val Arg Lys Asn Asp Gln Val
                1155 1160 1165
Tyr Ile Asn Phe Val Ala Ser Lys Thr His Leu Phe Pro Leu Tyr Ala
        1170 1175 1180
Asp Thr Ala Thr Thr Asn Lys Glu Lys Thr Ile Lys Ile Ser Ser Ser
1185 1190 1195 1200
Gly Asn Arg Phe Asn Gln Val Val Val Met Asn Ser Val Gly Asn Asn
                                1205 1210 1215
Cys Thr Met Asn Phe Lys Asn Asn Asn Gly Asn Asn Ile Gly Leu Leu
                        1220 1225 1230
Gly Phe Lys Ala Asp Thr Val Val Ala Ser Thr Trp Tyr Tyr Thr His
                1235 1240 1245
Met Arg Asp His Thr Asn Ser Asn Gly Cys Phe Trp Asn Phe Ile Ser
        1250 1255 1260
Glu Glu His Gly Trp Gln Glu Lys
1265 1270
```

1. A botulinum neurotoxin subtype E with reduced persistence having a sequence according to SEQ ID NO: 1, or SEQ ID NO: 2 or a functionally active variant thereof, for use in the treatment of a patient, wherein the patient is suffering from a medical condition or overexertion, or has undergone a therapeutic intervention, wherein said medical conditions, or said therapeutic intervention, respectively, results in a muscular disorder and/or injury that requires paralysis of the muscle or muscles involved in said disorder and/or injury.

2. The botulinum neurotoxin subtype E with reduced persistence for use in the treatment according to claim 1, which is a functionally active variant of a botulinum neurotoxin subtype E with reduced persistence having a sequence according to SEQ ID NO: 1, or SEQ ID NO: 2, wherein said functionally active variant has a persistence that is at maximum 5% shorter or longer than the duration of paralysis achieved by a botulinum neurotoxin subtype E with reduced persistence having a sequence according to SEQ ID NO: 1 or SEQ ID NO: 2.

3. The botulinum neurotoxin subtype E with reduced persistence for use in the treatment of claim 1, wherein said medical condition or overexertion, or said therapeutic intervention mandates a full physiotherapeutic treatment of said patient within less than four weeks.

4. The botulinum neurotoxin subtype E with reduced persistence for use in the treatment according to claim 1, wherein said medical condition is selected from the list of: (a) pre- or post-surgical local muscle relaxation that improves or accelerates restitution, (b) muscle contracture or contractions unrelated to surgery that benefit from transient local relaxation, and (c) pathologic muscular of connective tissue conditions that can be treated only or earlier by active or passive physical therapy.

5. The botulinum neurotoxin subtype E with reduced persistence for use in the treatment of claim 1, wherein said patient is a patient suffering from involuntary muscle movements associated with a disease.

6. The botulinum neurotoxin subtype E with reduced persistence for use in the treatment of claim 5, wherein said involuntary muscle movements associated with a disease as PD or any other disease or disorder accompanied by involuntary muscle movements, optionally tremor.

7. The botulinum neurotoxin subtype E with reduced persistence for use in the treatment of claim 1, wherein muscle paralysis by a botulinum neurotoxin of more than 5 weeks, optionally more than 4 weeks, and optionally more than 3 weeks, is contraindicated and/or deemed to be associated with negative impact on overall treatment success, optionally due to high likelihood of increased muscle atrophy.

8. The botulinum neurotoxin subtype E with reduced persistence for use in the treatment of claim 1, wherein said patient is suffering from a medical condition selected from the list of: overexertion (muscle fatigue), muscle tightness, increased and/or imbalanced muscle tonus, muscle contraction, muscle cramps, acute spasms, or muscle contracture, a muscle imbalance between an agonist and antagonist muscle and pathologies resulting therefrom.

9. The botulinum neurotoxin subtype E with reduced persistence for use in the treatment of claim 1, wherein said patient is suffering from a muscle injuries selected from a muscle strain, muscle tear, and a wound.

10. The botulinum neurotoxin subtype E with reduced persistence for use in the treatment of claim 1, wherein said patient is suffering from a foot or ankle pathology.

11. The botulinum neurotoxin subtype E with reduced persistence for use in the treatment of claim 10, wherein said patient is suffering from a pathology caused by a contracture of the musculus gastrocnemius and/or the musculus soleus.

12. The botulinum neurotoxin subtype E with reduced persistence for use in the treatment of claim 10, wherein said patient is suffering from a debilitating foot condition, in particular selected from the list of: flat foot, metatarsalgia, capsulitis, plantar fasciitis, hallux valgus, curled fifth toe, pes equinus, sesamoiditis, diabetic foot ulcers, Achilles tendon inflammation, tendinitis, Achilles tendon rupture, pes equinus, pes cavus, hammer toe, idiopathic toe walking and charcot athropathy.

13. A product comprising a botulinum neurotoxin subtype E with reduced persistence having a sequence according to SEQ ID NO: 1 or SEQ ID NO: 2, or a functionally active variant thereof, for use in the treatment of a patient, wherein the patient is suffering from a medical condition or overexertion, or has undergone a therapeutic intervention, wherein said medical conditions, or said therapeutic intervention, respectively, results in a muscular disorder and/or injury that requires paralysis of the muscle or muscles involved in said disorder and/or injury.

14. A method for treating a patient suffering from a medical condition or overexertion, or has undergone a therapeutic intervention, wherein said medical conditions, or said therapeutic intervention, respectively, results in a muscular disorder and/or injury that requires paralysis of the muscle or muscles involved in said disorder and/or injury, said method comprising administering an effective amount of a botulinum neurotoxin subtype E with reduced persistence having a sequence according to SEQ ID NO: 1 or SEQ ID NO: 2, or a functionally active variant thereof.


SEQUENCE LISTING (2 sequences)

SEQ ID NO: 1 - protein, 1272 aa, Artificial Sequence
  recombinant botulinum neurotoxin subtype E with reduced persistence with MDM2 binding motif
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSLTFEHNWAQLENKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 2 - protein, 1272 aa, Artificial Sequence
  botulinum neurotoxin subtype E with reduced persistence
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSLTFEHNWAQLTSKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK